(19) United States
(12) Patent Application Publication
(10) Pub. No.: US 2004/0010189 A1
(43) Pub. Date: Jan. 15, 2004

(54) Guide wire

(75) Inventors: Math J.J. van Sloun, Berg aan de Maas (NL); Dick Vos, Dalfsen (NL); Cornelius Carolus de Cock, Haarlem (NL)

Correspondence Address: HAHN LOESER & PARKS, LLP, TWIN OAKS ESTATE, 1225 W. MARKET STREET, AKRON, OH, 44313, US

(73) Assignee: Biotronik Mess-und Therapiegeraete GmbH & Co. Ingenieurbuero Berlin

(21) Appl. No.: 10/334,046

(22) Filed: Dec. 30, 2002

(30) Foreign Application Priority Data
Jul. 2, 2001 (DE) 101 32 332.1

(51) Int. Cl. A61B 5/04

(52) U.S. Cl. 600/374; 607/122; 600/585

(57) ABSTRACT

A guide wire (1) is provided which can be introduced intravascularly and which is used for implanting a catheter in a heart with an electrode (2, 5). In that arrangement, the guide wire (1) is first used as a temporary electrode (2, 5) for stimulating, defibrillating and/or sensing the heart in order to determine the optimum implantation position for the catheter to be introduced. When that optimum position is determined, the guide wire (1) is left at that position and the catheter is introduced along the guide wire (1) to the ascertained position and implanted. Once the catheter is implanted, the guide wire (1) is removed.

The present invention concerns a controllable guide wire, in particular for use during implantation of catheters in a heart, wherein the guide wire is adapted to be introduced intravascularly so that a catheter can then be introduced for implantation purposes along the guide wire, and wherein the guide wire is adapted to be removed again after implantation of the catheter.

### BACKGROUND OF THE ART

In the case of patients with pronounced heart failure, stimulation of the heart can prove to be advantageous. However, just in relation to a small group (with a long PR-interval), stimulation in the right ventricle resulted in significant improvements. Other stimulation arrangements were investigated for modification of the left ventricular function of the heart. It emerged that optimum stimulation of the heart in regard to the hemodynamic state of the heart appears to be different for each patient.

Therefore, to establish an optimum stimulation arrangement for the patient, it is absolutely necessary for the heart of the patient to be firstly investigated with regard to optimum therapy and an optimum arrangement of the respective electrodes, in regard to the desired hemodynamic values.

U.S. Pat. No. 5,549,109, to Samson, discloses a catheter and a guide wire for reproducing the coronary electrical activity in the coronary arteries and/or veins, wherein both the catheter and also the guide wire have electrodes. The guide wire and the catheter can be introduced from an external location, such as, for example, the femoral artery or vein, into the cardiac arteries or veins. The guide wire and the catheter are placed there in such a way that the local cardiac electrical activity in the cardiac muscle wall is measured and monitored. A complete image of the electrical activity of the heart can be formed by the variation in the measurement locations in the coronary vascular system.

When a complete image of the electrical activity of the heart has been produced the catheter together with the guide wire is removed from the patient again so that an electrical therapy device can be permanently implanted with a suitable catheter for stimulation and/or defibrillation of the heart. In that respect, the operation of introducing the catheter to the desired position is found to be highly complex as inaccurate placement has an adverse effect on the stimulation and defibrillation properties. In that respect it is in particular very difficult to precisely find the optimum placement for the catheter again.

Therefore the object of the invention is to provide a guide wire with which the placement of a catheter to be introduced can be improved.

### SUMMARY OF THE INVENTION

That object is attained by a guide wire of the kind set forth in the opening part of this specification, with the characterizing features of accompanying claim 1.

In that respect the invention is based on the idea of providing a guide wire which can be introduced intravascularly and which is used for the implantation of a catheter into a heart with an electrode. In that case the guide wire is firstly used as a temporary electrode for stimulation, defibrillation and/or sensing of the heart in order to determine the optimum implantation position for the catheter to be introduced. When that optimum position is determined the guide wire is left at that position and the catheter is introduced along the guide wire to the ascertained position and implanted. As soon as the catheter has been implanted the guide wire is removed again.

The advantages that the invention entails are that the optimum implantation position for the catheter to be implanted is ascertained by a guide wire with the electrode, and that the guide wire is left at that position and the catheter is introduced along the guide wire to that position. This ensures that the catheter is implanted at the precise ascertained position, thereby achieving optimum stimulation and defibrillation properties.

In a preferred configuration of the invention, the guide wire is provided with a single electrode which is preferably disposed at the distal end of the guide wire.

In a further embodiment of the invention, X-ray markers are provided on the guide wire. Those X-ray markers serve to make the, ascertained implantation position for the catheter to be implanted visible even outside the body so that the doctor carrying out the implantation operation can monitor the implantation procedure and finds the ascertained implantation position again more easily.

In still a further embodiment of the invention, a mechanical or a magnetic abutment is mounted to the distal end of the guide wire so that it can indicate the end of the guide wire and the doctor carrying out the implantation operation does not push the catheter beyond the distal end of the guide wire and thus miss the optimum implantation position.

In a further preferred embodiment of the invention, an optical marking is provided at the proximal end of the guide wire so that it is possible to perceive when the catheter to be introduced has been advanced to the ascertained implantation position. That optical marking also serves to provide that the catheter is not pushed beyond the distal end of the guide wire and thus misses the optimum position.

In a further particularly preferred embodiment of the invention, the guide wire is of a diameter of between 0.4 and 0.8 mm. That diameter range ensures that the guide wire can move to all desired positions in the cardiovascular system.

Further configurations according to the invention are the subject matter of the appendant claims.

### BRIEF DESCRIPTION OF THE DRAWINGS

Better understanding of the present invention will be obtained from reference to the accompanying drawings, in which identical parts are identified with identical reference numbers and in which:

FIG. 1 shows a view in section of a guide wire, and

FIG. 2 shows a cross-section at the distal end of the guide wire in FIG. 1.

### DETAILED DESCRIPTION OF THE PREFERRED EMBODIMENT

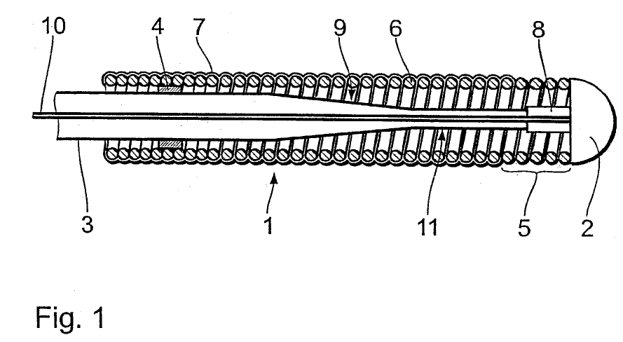

FIG. 1 shows a sectional view of a guide wire 1. The guide wire 1 has a helical coil 6 that extends as far as the distal end of a shank 3. At its distal end the shank 3 is connected to a rounded tip 2 and also by way of a solder join 4 to the coil 6.

The shank 3 in wire form is coated with a polytetrafluoroethylene coating, such as TEFLON, and is provided distally in relation to a tapering region 9 with a region 11 of reduced width and a wider region 8 at the distal end of the shank which is of a pressed-flat configuration and joined to the rounded tip 2. By virtue of the shank 3 being pressed flat at its distal end, that is to say in the region 8, the transmission of torsion is increased and at the same time the risk of fatigue is reduced.

At its outside surface the guide wire 1 has substantially—with the exception of the portion 5—a TEFLON coating 7 that is used as insulation. That coating also serves to reduce the friction of the guide wire. The non-insulated portion 5 is used as an electrode, the electrical contacting of which is effected by way of the coil 6.

The turns of the coil 6 are slightly opened at the distal end thereof, that is to say in the region of the portion 5, in order to increase the flexibility of the distal end of the guide wire 1.

The guide wire 1 also has a control wire 10 that is fixed at its distal end to the rounded tip 2 and which extends as far as the proximal end of the guide wire 1.

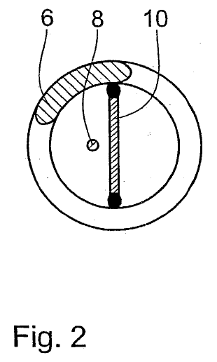

FIG. 2 shows a cross-section through the guide wire 1 in its distal region. The flat-pressed distal end 8 of the shank 3 and the distal end of the control wire 10 are in this case fixed in the rounded tip 2. The coil 6 is arranged around the shank 3 and the control wire 10.

When the control wire 10 is actuated or pulled in the proximal direction the distal end of the guide wire 1 experiences a flexing effect in accordance with the extent of actuation. The control wire 10 is suitably actuated for maneuvering in arteries and veins so that the distal end of the guide wire 1 flexes and the guide wire can thus be advanced around a bend or into a further artery or vein. As the guide wire 1 can only be flexed in one direction by means of the control wire 10 it is necessary for the entire guide wire to be rotated if the distal end of the guide wire 1 is to be flexed in a different direction. That is effected by rotating the shank 3 which is connected to the coil 6 by way of the solder join 4 and to the rounded tip 2.

As described hereinbefore the guide wire 1 is maneuvered through arteries and veins in order to pass into the atrium or the ventricle. When the distal end of the guide wire 1 has reached the desired location in the atrium or ventricle the non-insulated portion 5 is used as a monopolar electrode. In that case the monopolar electrode 5 is used as a temporary electrode in order to sense, stimulate and/or defibrillate the tissue therearound. The results of sensing, stimulation and defibrillation at that position are recorded. That recording procedure is carried out for a plurality of positions in the atrium and in the ventricle in order to find the optimum position for a catheter to be implanted for sensing, stimulation or defibrillation purposes.

When the optimum position is found the distal end of the guide wire is left at that position and the catheter to be introduced is introduced along the guide wire 1 to the distal end of the guide wire 1 (“over the wire” or “monorail”). As soon as the catheter has been introduced and implanted the guide wire 1 is removed again.

The guide wire 1 thus only serves to find an optimum implantation position and to introduce a catheter to be implanted, to the optimum implantation position found. The guide wire 1 is not intended for permanent implantation.

The guide wire can be positioned for example in the region of the coronary sinus in order to stimulate the left ventricle.

As an alternative to using the non-insulated portion 5 as a monopolar electrode it is also possible to use the rounded tip 2 of the guide wire 1 as an electrode, preferably as a tip electrode. For that purpose the portion 5 is provided with an insulating material and the insulation is removed around the rounded tip 2 of the guide wire 1. In that case the rounded tip 2 can be electrically contacted by way of the shank 3.

The diameter of the guide wire 1 is in the diameter range of between 0.4 and 0.8 mm.

Provided at the distal end of the guide wire 1 is an X-ray marker, preferably comprising gold. As an alternative thereto, the rounded tip 2 can be made from gold and can thus serve as an X-ray marker. The optimum implantation position ascertained by the guide wire 1 as a temporary electrode can be rendered visible by means of such an X-ray marker in order then to be able to move exactly to that position with an electrode catheter which is also provided with an X-ray marker.

As an alternative thereto, it is possible to envisage a mechanical marking in the form of an abutment at the distal end of the guide wire 1, which provides that, upon being inserted to the optimum implantation position ascertained, the catheter is not unintentionally introduced beyond the distal end of the guide wire 1 and thus misses the optimum implantation position. In that case however the abutment is not to be designed in such a way that it is insurmountable as otherwise the guide wire would no longer be removable after the implantation operation. As an alternative to the mechanical abutment a magnetically perceptible abutment would also be possible. A magnetic abutment of that kind could be implemented for example by using magnets at the distal end of the guide wire and at the distal end of the catheter to be implanted.

A further alternative way of preventing the catheter being introduced beyond the distal end of the guide wire 1 represents an optical marking on the guide wire and/or the catheter respectively in the proximal region thereof, that is to say outside the body of the patient. By reference to an optical marking of that kind it is possible for the operator of the guide wire 1 and the catheter to recognize when the distal end of the catheter to be introduced has reached the distal end of the guide wire so that the catheter to be introduced is thus at the desired implantation position and can be implanted.

What is claimed is:

1. A controllable guide wire, especially for use in implanting a catheter in a heart, wherein the guide wire has a proximal and a distal end and is adapted to be introduced intravascularly so that the catheter may be introduced for implantation therealong and wherein the guide wire is adapted to be removed after implanting the catheter, comprising:
at least one electrode fixed to the guide wire, said at least one electrode being adapted, when the guide wire is introduced, to stimulate, defibrillate and/or sense adjoining tissue in order to determine an optimum intravascular implantation location for the cathode to be implanted,
wherein the guide wire is adapted to introduce the catheter to be implanted along the guide wire to the implantation location determined by said at least one electrode.

2. The guide wire of claim 1, comprising a single electrode.

3. The guide wire of claim 2, wherein the electrode is at the distal end of the guide wire.

4. The guide wire of claim 1, further comprising:
at least one X-ray marker on the guide wire, the marker being adapted to make the ascertained implantation position visible outside the body.

5. The guide wire of claim 2, further comprising:
at least one X-ray marker on the guide wire, the marker being adapted to make the ascertained implantation position visible outside the body.

6. The guide wire of claim 3, further comprising:
at least one X-ray marker on the guide wire, the marker being adapted to make the ascertained implantation position visible outside the body.

7. The guide wire of claim 1, further comprising:
a mechanical abutment at the distal end of the guide wire, the abutment being adapted to indicate the distal end of the guide wire.

8. The guide wire of claim 2, further comprising:
a mechanical abutment at the distal end of the guide wire, the abutment being adapted to indicate the distal end of the guide wire.

9. The guide wire of claim 5, further comprising:
a mechanical abutment at the distal end of the guide wire, the abutment being adapted to indicate the distal end of the guide wire.

10. The guide wire of claim 1, further comprising:
a magnetic, perceptible abutment at the distal end of the guide wire, the abutment being adapted to indicate the distal end of the guide wire.

11. The guide wire of claim 2, further comprising:
a magnetic, perceptible abutment at the distal end of the guide wire, the abutment being adapted to indicate the distal end of the guide wire.

12. The guide wire of claim 9, further comprising:
a magnetic, perceptible abutment at the distal end of the guide wire, the abutment being adapted to indicate the distal end of the guide wire.

13. The guide wire of claim 1, further comprising:
an optical marking at the proximal extracorporal end of the guide wire, the optical marking being adapted be visible when the catheter to be introduced has been advanced as far as the ascertained implantation location.

14. The guide wire of claim 2, further comprising:
an optical marking at the proximal extracorporal end of the guide wire, the optical marking being adapted be visible when the catheter to be introduced has been advanced as far as the ascertained implantation location.

15. The guide wire of claim 4, further comprising:
an optical marking at the proximal extracorporal end of the guide wire, the optical marking being adapted be visible when the catheter to be introduced has been advanced as far as the ascertained implantation location.

16. The guide wire of claim 7, further comprising:
an optical marking at the proximal extracorporal end of the guide wire, the optical marking being adapted be visible when the catheter to be introduced has been advanced as far as the ascertained implantation location.

17. The guide wire of claim 10, further comprising:
an optical marking at the proximal extracorporal end of the guide wire, the optical marking being adapted be visible when the catheter to be introduced has been advanced as far as the ascertained implantation location.

18. The guide wire of claim 12, further comprising:
an optical marking at the proximal extracorporal end of the guide wire, the optical marking being adapted be visible when the catheter to be introduced has been advanced as far as the ascertained implantation location.

19. The guide wire of claim 1, wherein the guide wire has a diameter between 0.4 and 0.8 mm.

20. The guide wire of claim 18, wherein the guide wire has a diameter between 0.4 and 0.8 mm.

21. The guide wire of claim 1, wherein a wire coil provides the electrical feed for said at least one electrode of the guide wire.

22. The guide wire of claim 2, wherein a wire coil provides the electrical feed for the single electrode of the guide wire.

23. The guide wire of claim 20, wherein a wire coil provides the electrical feed for the single electrode of the guide wire.

24. The guide wire of claim 1, wherein a shank provides the electrical feed for the said at least one electrode of the guide wire.

25. The guide wire of claim 2, wherein a shank provides the electrical feed for the single electrode of the guide wire.

26. The guide wire of claim 20, wherein a shank provides the electrical feed for the said at least one electrode of the guide wire.
